(19) United States
(12) Patent Application Publication
(10) Pub. No.: US 2003/0195139 A1
(43) Pub. Date: Oct. 16, 2003

(54) Metabotropic glutamate receptor antagonists for treating tolerance and dependency

(76) Inventors: Mauro Corsi, Verona (IT); Francois Conquet, Laussane (CH)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/221,128

(22) PCT Filed: Mar. 9, 2001
(86) PCT No.: PCT/GB01/01058

(30) Foreign Application Priority Data
Mar. 9, 2000 (GB) 0005700.0

(51) Int. Cl. A61K 31/00

(52) U.S. Cl. 514/1

(57) ABSTRACT

An antagonist of the metabotropic glutamate receptor 5 (mGluR5) is useful in tolerance or dependence therapy. Such an antagonist can therefore be used in the treatment of substance tolerance or dependence, bulimia nervosa, anorexia nervosa, gambling dependence, sex dependence or obsessive compulsive disorders.

### FIELD OF THE INVENTION

The invention relates to tolerance and dependence therapy. It also relates to screening methods for identifying new products which can be used in tolerance and dependence therapy.

### BACKGROUND TO THE INVENTION

Addiction is a chronic brain disease manifested by humans in a variety of behaviours and in a range of social circumstances. Although it is a complex phenomenon, its medical definition is a central nervous system (CNS) disorder manifested as a behavioural disturbance due to a neurobiological imbalance in the brain (Leshner, 1997, Science 278, 45). Individuals may become addicted to a wide variety of factors, including substances like drugs. The obsessive and compulsive aspect of drug dependence may overlap with other obsessive compulsive behaviours such as gambling or compulsive sexual activity.

In respect of substance abuse, addict behaviour is induced and maintained in a multifactorial fashion with a central role played by the unconditioned reinforcing properties of the abused drug. There are many different substances on which individuals may become dependent, including opiates, benzodiazepines, amphetamine, nicotine, cocaine and ethanol.

The impact of substance dependence is huge. For example, nicotine dependence is the most widely diffused type of drug addiction. One third of the worldwide population over 15 years of age are smokers. Smoking continues to increase among adolescents and by the year 2025 the WHO estimates that there will be 10 million tobacco related deaths per year. Stopping smoking may evoke a range of symptoms in dependent individuals, including craving, depression, anxiety, difficulty in concentrating and weight gain. Despite a variety of available treatments many smokers fail to give up smoking.

There is therefore a major unmet need in the area of substance abuse for pharmacological agents that are more effective that those currently available at reducing withdrawal symptoms and more importantly reducing relapse rates. Indeed smoking cessation is a therapeutic area with generally poor results: an avearage 30% success rate compared with 50 to 80% for alcoholism, opioid and cocaine dependence (at 6 months).

Nevertheless the rationale for pharmacological intervention is, however, still strong because only pharmacotherapy potentially acts on a population larger than that treated with psychosocial interventions and therefore may enhance these traditional methods by improving compliance and quality of the treatment.

Glutamate is the transmitter of the vast majority of excitatory synapses in the mammalian CNS and plays an important role in a wide variety of CNS functions. In the past, the actions of glutamate in the mammalian brain were thought to be mediated exclusively by activation of glutamate-gated cation channels termed ionotropic glutamate receptors: see Watkins & Evans, Ann. Rev. Pharmacol. Toxicol., 21, 165 (1985). In the mid-1980s, however, evidence for the existence of glutamate receptors directly coupled to a second messenger via G-proteins began to appear with the discovery of a glutamate receptor coupled to activation of phosphonoinositide hydrolysis. That led to the discovery of a new family of glutamate receptors named metabotropic glutamate receptors (mGluRs): see for example. Sladeczek et al. Nature. 317, 717 (1985) and Sugiyama et al., Nature, 325, 531 (1987).

The search for mGluR-related cDNAs has resulted in the isolation of eight genes that encode distinct mGluRs. These receptors are named mGluR1 through mGluR8. Based on their amino acid sequence identity the eight mGluRs can be classified into three groups. Group I includes mGluR1 and mGluR5, group II mGluR2 and mGlu and group III mGluR4, mGluR6, mGluR7 and mGluR8. Whereas group I mGluRs stimulate inositol phosphate metabolism and mobilization of intracellular Ca²⁺, both group II and group III mGluRs are negatively coupled to adenylyl cyclase (Schoepp & Conn, Trend Pharmacol. Sci., 14, 13, 1993 and Pin & Duvoisin, Neuropharmacology, 34, 1 (1995)).

### SUMMARY OF THE INVENTION

This invention is based on our findings that:

(i) a compound which can act as a selective antagonist of the metabotropic glutamate receptor, mGluR5, can reduce the reinstatement of nicotine-seeking behaviour in rats following exposure to experimental determinants of smoking relapse; and

(ii) mGluR5 knockout mice do not display cocaine-induced hyperactivity and show no response to the reinforcing properties of cocaine and do not self-administer amphetamine at any tested dose.

We propose that pharmacological block of mGluR5 leads to negative regulation of dopamine-2 (D2) receptors, which in turn reduces dopaminergic activity. The reduction in dopaminergic activity leads to a reduction in smoking relapse. We also propose that mGluR5 is responsible for the hyperactive response to cocaine administration and is also an essential component of the reward process induced by cocaine and amphetamine.

Furthermore, we propose that mGluR5 is involved in “emotional learning”. Addiction implies that an individual has first “learned” how to be dependent before being addicted. Every memorisation process is preceded and we suggest that mGluR5 is responsible for the “learning” process of dependence, regardless of the type of dependence in question. The mGluR5 receptor is thus required for the onset of the dependence process, before the final settlement of physiological addiction.

According to the present invention there is thus provided use of an antagonist of mGluR5, typically human mGluR5, in the manufacture of a medicament for use in a method of tolerance or dependence therapy.

The invention also provides:

an antagonist of mGluR5 for use in a method of treatment of the human or animal body by therapy;

a method of treating a host suffering from tolerance or dependence, which method comprises administering to the host a therapeutically effective amount of an antagonist of mGluR5;

a pharmaceutical composition comprising an antagonist of mGluR5 and a pharmaceutically acceptable carrier or diluent;

products containing an antagonist of mGluR5 and a therapeutic substance as a combined preparation for simultaneous, separate or sequential use in the treatment of a condition for which the said therapeutic substance is used, wherein the use of the therapeutic substance in the absence of said antagonist could lead to tolerance of or dependence on the therapeutic substance;

use of mGluR5 for identifying a product for use in the treatment tolerance or dependence;

a method for identifying a product for use in the treatment of tolerance or dependence, comprising:

(a) contacting a test product with mGluR5 under conditions that in the absence of the test substance would lead to activity of the said mGluR5 and

(b) determining whether the test product antagonises mGluR5 activity, thereby to determine whether the test product may be used in the treatment of substance tolerance or dependence;

a product identified by a method of the invention;

a product of the invention for use in a method of treatment of the human or animal body by therapy;

use of a product of the invention for the manufacture of a medicament for use in tolerance or dependence therapy;

a method of treating a host suffering from tolerance or dependence, which method comprises administering to the host a therapeutically effective amount of a product of the invention;

a pharmaceutical composition comprising a product of the invention and a pharmaceutically acceptable carrier or diluent; and

products containing a product of the invention and a therapeutic substance as a combined preparation for simultaneous, separate or sequential use in the treatment of a condition for which the said therapeutic substance is used, wherein the use of the therapeutic substance in the absence of said product could lead to tolerance of or dependence on the therapeutic substance.

### BRIEF DESCRIPTION OF THE FIGURES

FIG. 1(a) shows the effects of cocaine on mutant (●) and wild-type mice (▪) mice motor activity. Horizontal activity was measured every 5 minutes during a 60-min session. Mice were injected with saline i.p. and placed in the apparatus at time 0. At time 15 min, mice were injected with cocaine 10 mg/kg i.p. and placed again in the apparatus. Statistics (oneway ANOVA; n=5-12) was performed by comparison of values from time bin 20 to 60 min. SEM are omitted.

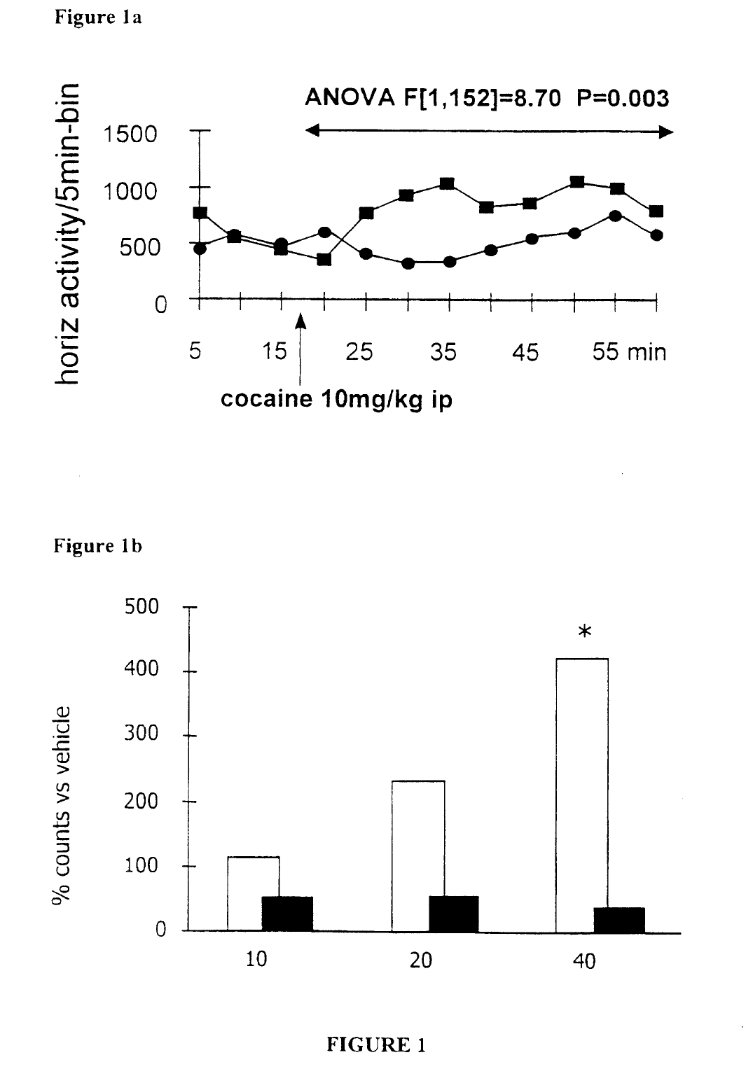

(b) shows the effects of cocaine on mutant (filled bars) and wild-type mice (open bars) motor activity. The total amount of horizontal activity measure during the 60-min session was calculated as percentage of vehicle treatment effect (% counts vs vehicle). Mice were injected with saline i.p. and placed in the apparatus at time 0. At time 15 min. mice were injected with cocaine 10, 20 or 40 mg/kg i.p. and placed again in the apparatus. Statistics (oneway ANOVA followed by Dunnett's; *=P<0.05; n=14-16) were performed by comparison of values from time bin 20 to 60 min. SEM are omitted.

FIG. 2 shows the results of cocaine self-administration experiments;

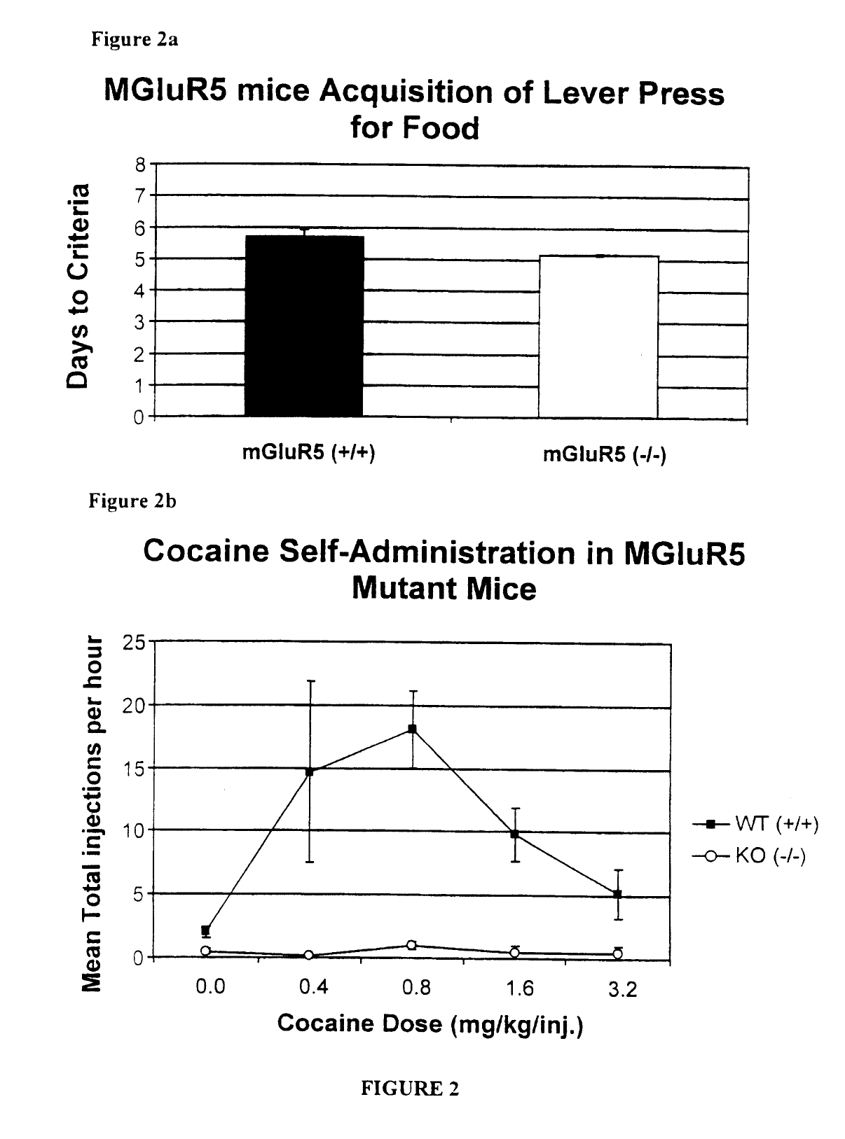

(a) Learing task experiment with food reinforcer (sugar milk) shows that both mGlu(+/+) (filled bar) and (−/−) (open bar) performed an equal operant behaviour; and

(b) Dose response curve at different doses of cocaine in self-administration paradigm for wild type (▪) and knockout (O)mice.

FIG. 3 shows the results of dopamine microdialysis in the nucleus accumbens of (a) wild type and (b) knockout mice after injection of 10 mg/kg of cocaine or saline buffer intraperiteonally.

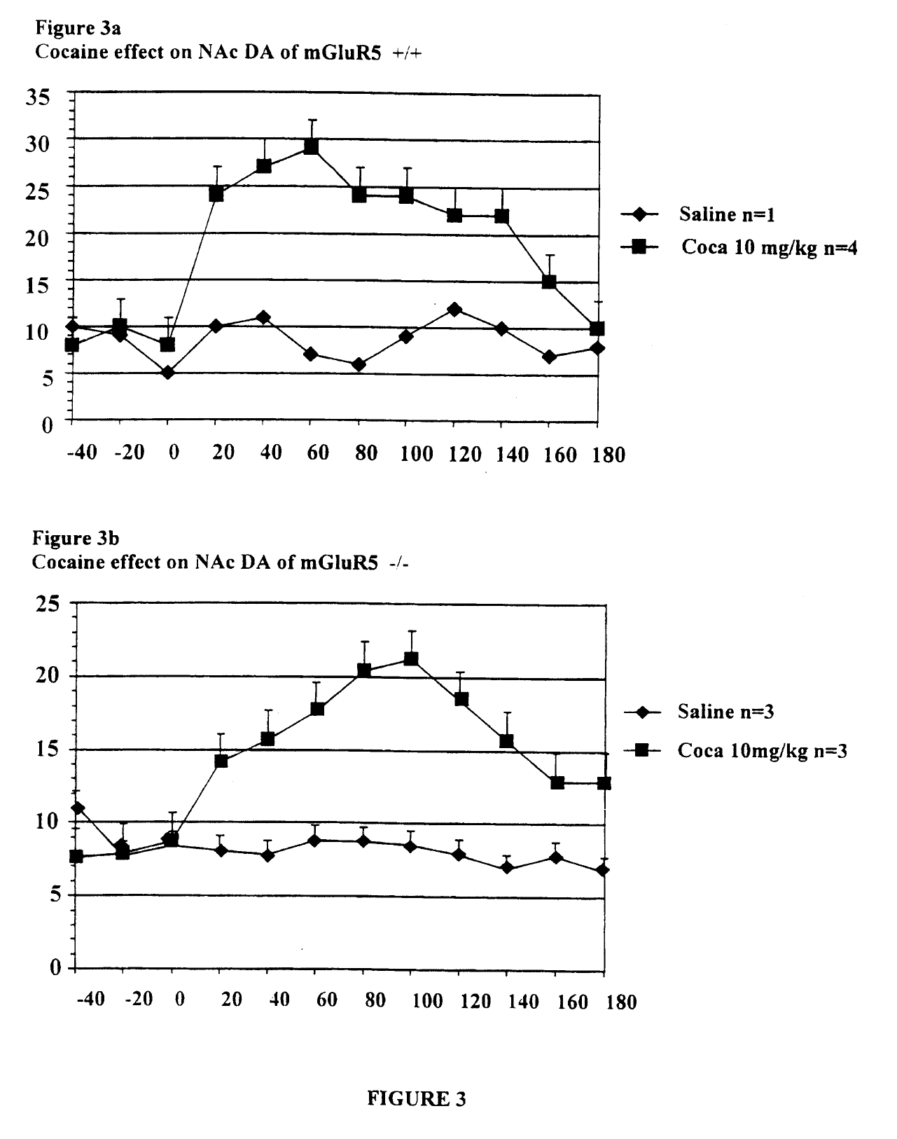

FIG. 4 shows d-amphetamine self-administration in mGluR5 knock-out (−/−) mice.

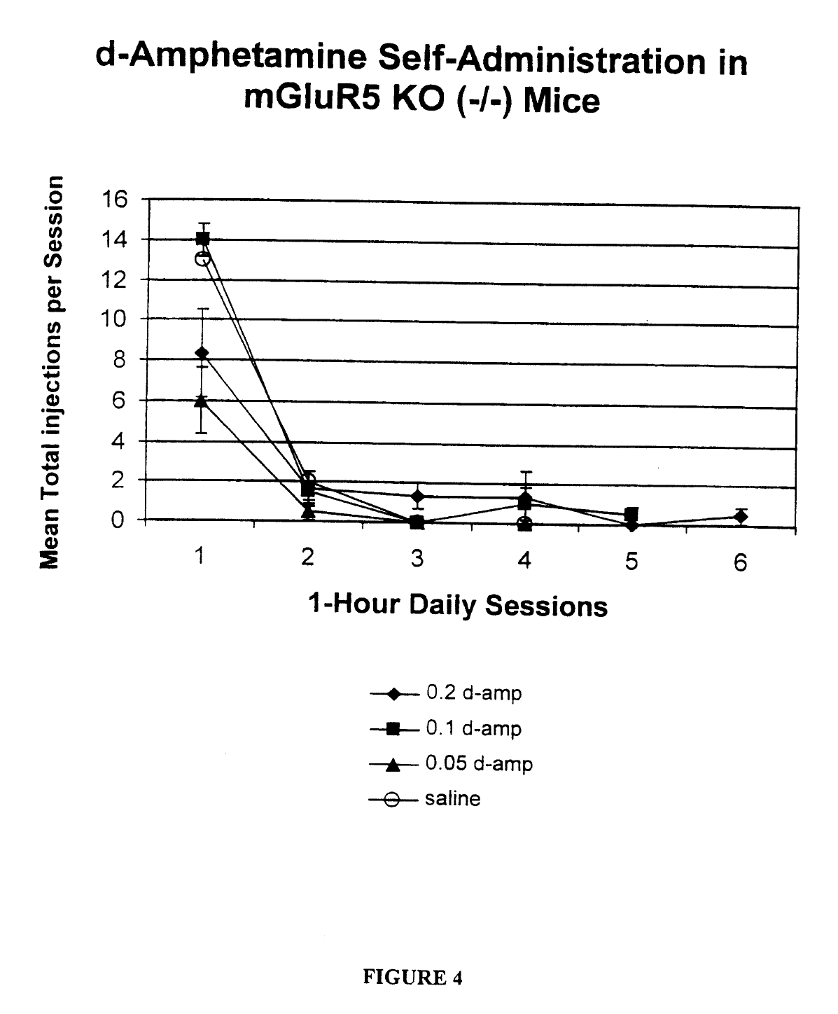

### DETAILED DESCRIPTION OF THE INVENTION

The present invention is concerned with antagonists of mGluR5 for treating tolerance or dependence. The mGluR5 is preferably human mGluR5.

An antagonist of mGluR5 is a substance which diminishes or abolishes the effect of a ligand (agonist) which typically activates mGluR5. Thus, the antagonist may be, for example, a chemical antagonist, a pharmacokinetic antagonist, an antagonist by receptor block, a non-competitive antagonist or a physiological antagonist.

A chemical antagonist is wherein the antagonist binds the ligand in solution so the effect of the ligand is lost.

A pharmacokinetic antagonist is one which effectively reduces the concentration of the active drug at its site of action, for example by increasing the rate of metabolic degradation of the active ligand.

Antagonism by receptor-block involves two important mechanisms: reversible competitive antagonism and irreversible, or non-equilibrium, competitive antagonism. Reversible competitive antagonism occurs when the rate of dissociation of the antagonist molecules is sufficiently high that, on addition of the ligand, displacement of the antagonist molecules from the receptors effectively occurs. Of course the ligand cannot evict a bound antagonist molecule, or vice versa. Irreversible or non-equilibrium, competitive antagonism occurs when the antagonist dissociates very slowly, or not at all, from the receptor with the result that no change in the antagonist occupancy takes place when the ligand is applied. Thus the antagonism is insurmountable.

Non-competitive antagonism describes the situation where the antagonist blocks at some point in the signal transduction pathway leading to the production of a response by the ligand.

Physiological antagonism is a term used loosely to describe the interaction of two substances whose opposing actions in the body tend to cancel each other out.

An antagonist can also be a substance which diminishes or abolishes expression of functional mGluR5. Thus an antagonist can be, for example, a substance which diminishes or abolishes expression of the gene encoding mGluR5, diminishes or abolishes translation of mGluR5 RNA, diminishes or abolishes post-translational modification of mGluR3 protein or diminishes or abolishes the insertion of mGluR5 into the cell membrane.

Preferred antagonists are those which lead to a reduction of activation by the ligand of at least 10%, at least 20%, at least 30%, at least 40%, at least 50%, at least 60%, at least 70%, at least 80%, at least 90%, at least 95% or at least 99% at a concentration of the antagonist of 1 μgml⁻¹, 10 μgml⁻¹, 100 μgml⁻¹, 500 μgml⁻¹, 1 mgml⁻¹, 10 mgml⁻¹, 100 mg ml⁻¹. The percentage antagonism represents the percentage decrease in activity of mGluR5 in a comparison of assays in the presence and absence of the antagonist. Any combination of the above mentioned degrees of percentage antagonism and concentration of antagonist may be used to define an antagonist of the invention, with greater antagonism at lower concentrations being preferred.

An antagonist for use in the invention may be a relatively non-specific antagonist which is an antagonist of mGluRs in general. Preferably, however, an antagonist antagonises only group I mGluRs. More preferably, an antagonist used in the invention is a selective antagonist of mGluR5. A selective antagonist of mGluR5 is one which antagonises mGluR5, but antagonises other mGluRs only weakly or substantially not at all. Most preferred antagonists are those which can selectively antagonise mGluR5 at low concentrations, for example those that cause a level of antagonism of 50% or greater at a concentration of 100 μgml⁻¹ or less.

Selective mGluR5 antagonists can thus typically exhibit at least 100 fold greater activity at an mGluR5 receptor than at an mGluR1 receptor, preferably at least 200 fold greater activity and most preferably at least 400 fold greater activity. They can display a high degree of selectivity and affinity as antagonists of the human and/or rat mGluR5.

Suitable antagonists for use in the invention are disclosed in EP-A-0807621, WO 99/02497, WO 00/20001 and WO 00/63166. As disclosed in WO 99/02497, therefore, suitable antagonists may thus have the formula (I):

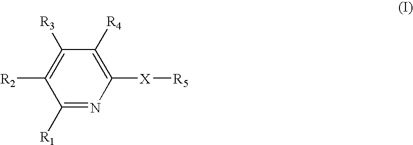

wherein

R₁ denotes hydrogen, lower alkyl, hydroxy-lower alkyl, lower alkyl-amino, piperidino, carboxy, esterified carboxy, amidated carboxy, unsubstituted or lower alkyl-, lower alkoxy-, halo- and/or trifluoromethyl-substituted N-lower-alkyl-CN-phenylcarbamoyl, lower alkoxy, halo-lower alkyl or halo-lower alkoxy;

R₂ denotes hydrogen, lower alkyl, carboxy, esterified carboxy, amidated carboxy, hydroxy-lower alkyl, hydroxy, lower alkoxy or lower alkanoyloxy, 4-(4-fluoro-benzoyl)-piperidin-1-ylcarboxy, 4-t.butyloxycarbonyl-piperazin-1-yl-carboxy, 4-(4-azido-2-hydroxybenzoyl)-piperazin-1-yl-carboxy or 4-(4-azido-2-hydroxy-3-iodo-benzoyl)-piperazin-1-yl-carboxy;

R₃ represents hydrogen, lower alkyl, carboxy, lower alkoxy-carbonyl, lower alkyl-carbamoyl, hydroxy-lower alkyl, di-lower alkyl-aminomethyl, morpholinocarbonyl or 4-(4-fluoro-benzoyl)-piperadin-1-yl-carboxy;

R₄ represents hydrogen, lower alkyl, hydroxy, hydroxy-lower alkyl, amino-lower alkyl, lower alkylamino-lower alkyl, di-lower alkylamino-lower alkyl, unsubstituted or hydroxy-substituted lower alkyleneamino-lower alkyl, lower alkoxy, lower alkanoyloxy, amino-lower alkoxy, lower alkylamino-lower alkoxy, di-lower alkylamino-lower alkoxy, phthalimido-lower alkoxy, unsubstituted or hydroxy- or 2-oxo-imidazolidin-1-yl-substituted lower alkyleneamino-lower alkoxy, carboxy, esterified or amidated carboxy, carboxy-lower alkoxy or esterified carboxy-lower alkoxy;

X represents an optionally halo-substituted lower alkenylene or alkynylene group bonded via vicinal saturated carbon atoms or an azo (—N═N—) group, and R₅ denotes an aromatic or heteroaromatic group which is unsubstituted or substituted by one or more substituents selected from lower alkyl, halo, halo-lower alkyl, halo-lower alkoxy, lower alkenyl, lower alkynyl, unsubstituted or lower alkyl-, lower alkoxy-, halo- and/or trifluoromethyl-substituted phenyl, unsubstituted or lower alkyl-, lower alkoxy-, halo and/or trifluoromethyl-substituted phenyl-lower alkynyl, hydroxy, hydroxy-lower alkyl, lower alkanoyloxy-lower alkyl, lower alkoxy, lower alkenyloxy, lower alkylenedioxy, lower alkanoyloxy, amino-, lower alkylamino-, lower alkanoylamino- or N-lower alkyl-N-lower alkanoylamino-lower alkoxy, unsubstituted or lower alkyl-, lower alkoxy-, halo- and/or trifluoromethyl-substituted phenoxy, unsubstituted or lower alkyl-, lower alkoxy-, halo and/or trifluoromethyl-substituted phenyl-lower alkoxy, acyl, carboxy, esterified carboxy, amidated carboxy, cyano, carboxy-lower alkylamino, esterified carboxy-lower alkylamino, amidated carboxy-lower alkylamino, phosphono-lower alkylamino, esterified phosphono-lower alkylamino, nitro, amino, lower alkylamino, di-lower alkylamino-acylamino, N-acyl-N-lower alkylamino, phenylamino, phenyl-lower alkylaamino, cycloalkyl-lower alkylamino or heteroaryl-lower alkylamino each of which may be unsubstituted or lower alkyl-, lower alkoxy-, halo- and/or trifluoromethyl-substituted; their N-oxides and their pharmaceutically acceptable salts.

Compounds of formula (I) which have basic groups may form acid addition salts and compounds of the formula (I) having acidic groups may form salts with bases. Compounds of formula (I) having basic groups and in addition having at least one acidic group, may also form internal salts. Also included are both total and partial salts, that is to say salts with 1, 2 or 3, preferably 2, equivalents of base per mole of acid of formula (I), or salts with 1, 2 or 3 equivalents, preferably 1 equivalent, of acid per mole of base of formula (I). Only the pharmaceutically acceptable, non-toxic salts are used therapeutically and they are therefore preferred.

Halo in the present description denotes fluorine, chlorine, bromine or iodine. Lower alkyl is typically C₁₋₆, for example C₁₋₄, alkyl. Lower alkoxy is typically C₁₋₆, for example C₁₋₄, alkoxy. Lower alkynyl is typically C₂₋₅ alkynyl. Lower alkanoyl is typically C₂₋₅alkanoyl. Lower alkylene is typically C₂₋₅ alkylene. Lower alkenylene is typically C₂₋₅ alkenylene. Lower alkynylene is typically C₂₋₄ alkynylene.

When X represents an alkenylene group, configuration trans is preferred.

Preferred compounds of formula (I) are those wherein:

X represents an optionally halo-substituted (C₂₋₄)alkenylene or alkynylene group bonded via vicinal unsaturated carbon atoms.

R₁ is hydrogen, (C₁₋₄) alkyl, (C₁₋₄)alkoxy, hydroxy(C₁₋₄)alkyl, cyano, ethynyl, carboxy, (C₁₋₄)alkoxycarbonyl, di(C₁₋₄)alkylamino, (C₁₋₆)alkylaminocarbonyl, trifluoromethylphenylaminocarbonyl,

R₂ is hydrogen hydroxy (C₁₋₄)alkyl, hydroxy (C₁₋₄)alkyl, (C₁₋₄)alkoxy, carboxy, (C₂₋₅)alkanoyloxy, (C₁₋₄)alkoxycarbonyl, di(C₁₋₄)alkylamino(C₁₋₄)alkanoyl, -di(C₁₋₄)alkylaminomethyl, 4-(4-fluoro-benzoyl)-piperidin-1-yl-carboxy, 4-t.-butyloxycarbonyl-piperazin-1-yl-carboxy, 4-(4-azido-2-hydroxybenzoyl)-piperazin-1-yl-carboxy or 4-(4-azido-2-hydroxy-3-iodo-benzoyl)-piperazin-1-yl-carboxy,

R₃ is hydrogen, (C₁₋₄) alkyl, carboxy, (C₁₋₄)alkoxycarbonyl, (C₁₋₄)alkyl-carbamoyl, hydroxy(C₁₋₄)alkyl, di(C₁₋₄)alkylaminomethyl, morpholinocarbonyl or 4-(4-fluoro-benzoyl)-piperidin-1-yl-carboxy,

R₄ is hydrogen, hydroxy, (C₁₋₄)alkoxy, carboxy, (C₂₋₅)alkanoyloxy, (C₁₋₄)alkoxy-carbonyl, amino(C₁₋₄)alkoxy, di(C₁₋₄)alkylamino(C₁₋₄)alkoxy, di(C₁₋₄)alkylamino(C₁₋₄)alkyl, carboxy (C₁₋₄)alkylcarbonyl, (C₁₋₄)alkoxycarbonyl(C₁₋₄ )alkoxy, hydroxy(C₁₋₄)alkyl, di(C₁₋₄)alkylamino(C₁₋₄)alkoxy, m-hydroxy-p-azidophenylcarbonylamino(C₁₋₄)alkoxy, and

R₅ is a group of formula

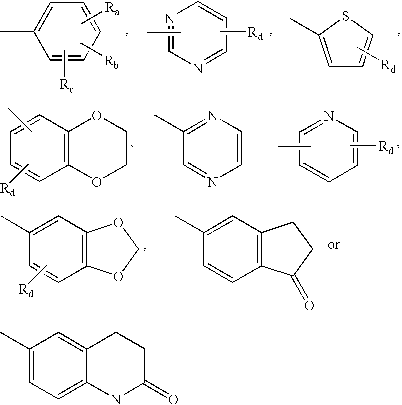

wherein:

Rₐ and R_{b} independently are hydrogen, hydroxy, halogen, nitro, cyano, carboxy, (C₁₋₄)alkyl (C₁₋₄)alkoxy, hydroxy(C₁₋₄)alkyl, (C₁₋₄)alkoxycarbonyl, (C₂₋₇ )alkanoyl, (C₂₋₅)alkanoyloxy, (C₂₋₅)alkanoyloxy(C₁₋₄)alkyl, trifluoromethyl, trifluoromethoxy, trimethylsilylethynyl, (C₂₋₅)alkynyl, amino, azido, amino(C₁₋₄)alkoxy, (C₂₋₅)alkanoylamino(C₁₋₄)alkoxy, (C₁₋₄)alkylamino(C₁₋₄)alkoxy, di(C₁₋₄)alkylamino(C₁₋₄)alkoxy, (C₁₋₄)alkylamino, di(C₁₋₄)alkylamino, monohalobenzylamino, thienylmethylamino, thienylcarbonylamino, trifluoromethylphenylaminocarbonyl, tetrazolyl, (C₂₋₅)alkanoymamino, benzylcarbonylamino, (C₁₋₄)alkylaminocarbonylamino. (C₁₋₄)alkoxycarbonyl-aminocarbonylamino or (C₁₋₄)alkylsulfonyl,

R_{c} is hydrogen, fluorine, chlorine, bromine, hydroxy, (C₁₋₄)alkyl, (C₂₋₅)alkanoyloxy, (C₁₋₄)alkoxy or cyano, and

R_{d} is hydrogen, halogen or (C₁₋₄)alkyl.

More preferred compounds of formula (I) are those wherein X is as defined above and

R₁ is hydrogen, (C₁₋₄)alkyl, (C₁₋₄)alkoxy, cyano, ethynyl or di(C₁₋₄)alkylamino,

R₂ is hydrogen, hydroxy carboxy, (C₁₋₄) alkoxycarbonyl, di(C₁₋₄)alkylaminomethyl, 4-(4-fluoro-benzoyl)-piperdin-1-yl-carboxy-4-t.-butyloxycarbonyl-piperizin-1-ylcarboxy, 4-(4-azido-2-hydroxybenzoyl)-pipeazin-1-yl-carboxy or 4-(4-azido-2-hydroxy-3-iodo-benzoyl)-piperazin-1-yl-carboxy,

R₃ is as defined above,

R₄ is hydrogen, hydroxy, carboxy, (C₂₋₅)alkanoyloxy, (C₁₋₄)alkoxycarbonyl, amino(C₁₋₄)alkoxy, di(C₁₋₄)alkylamino(C₁₋₄)alkoxy, di(C₁₋₄)alkylamino(C₁₋₄)alkyl or hydroxy(C₁₋₄)alkyl, and

R₅ is a group of formula

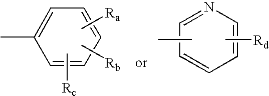

wherein:

Rₐ and R_{b} independently are hydrogen, halogen, nitro, cyano, (C₁,)alkyl, C(₁₋₄ )alkoxy, trifluoromethyl, trifluoromethoxy or (C₂₋₅)alkynyl, and R and Rd are as defined above.

Further selective mGluR antagonists are 2-arylalkenyl-, 2-heteroarylalkenyl-, 2-arylalkynyl-, 2-heteroaryl-alkynyl, 9-arylazo- and 2-heteroarylazo-pyridines, more particularly 6-methyl-2-(phenylazo)-3′-pyridinol, (E)-2-methyl-6-styryl-pyridine and -(phenylazo)-3-pyridinol, (E)-2-methyl-6-styryl-pyridine and compounds of formula (II):

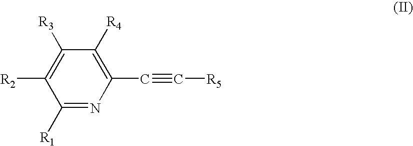

wherein

R₁ is hydrogen, (C₁₋₄)alkyl, (C₁₋₄)alkoxy, cyano, ethynyl or di(C₁₋₄)alkylamino,

R₂ is hydrogen, hydroxy, carboxy, (C₁₋₄) alkoxycarbonyl, di(C₁₋₄)alkylaminomethyl, 4-(4-fluoro-benzoyl)-piperidin-1-yl-carboxy, 4-t.butyloxycarbonyl-piperazin-1-yl-carboxy, 4-(4-azido-2-hydroxybenzoyl)-piperazin-1-yl-carboxy or 4-(4-azido-2-hydroxy-3-iodo-benzoyl)-piperazin-1-yl-carboxy,

R₃ is hydrogen, (C₁₋₄)alkyl, carboxy, (C₁₋₄)alkoxycarbonyl, (C₁₋₄)alkylcarbamoyl, hydroxy(C₁₋₄)alkyl, di(C₁₋₄)alkylaminomethyl, morpholinocarbonyl or 4-(4-fluoro-benzoyl)-piperazin-1-yl-carboxy,

R₄ is hydrogen, hydroxy, carboxy, C(₂₋₅)alkanoyloxy, (C₁₋₄)alkoxycarbonyl, amino (C₁₋₄)alkoxy, di(C₁₋₄)alkylamino(C, ₄)alkoxy, di(C₁₋₄)alkylamino(C₁₋₄)alkyl or hydroxy(C₁₋₄)alkyl, and

R₅ is a group of formula

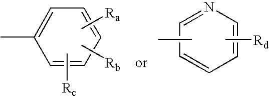

wherein

Rₐ and R_{b} independently are hydrogen, halogen, nitro, cyano, (C₁₋₄)alkyl, (C₁₋₄)alkoxy, trifluoromethyl, trifluoromethoxy or (C2,)alkynyl, and

R_{c} is hydrogen, fluorine, chlorine bromine, hydroxy (C₁₋₄)alkyl, C(₂₋₅)alkanoyloxy, (C₁₋₄)alkoxy or cyano; and

R_{d} is hydrogen, halogen or (C₁₋₄)alkyl;

in free form or in form of pharmaceutically acceptable salts.

Suitable phenyl glycine compounds are disclosed in EP-A-0807621. Phenyl glycine compounds useful in the invention can thus have the formula (III):

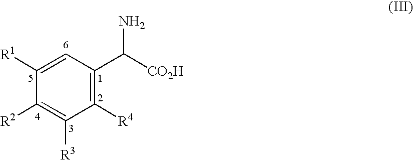

in which R¹ is hydrogen, hydroxy or C₁₋₆ alkoxy;

R₂ is hydrogen, carboxy, tetrazolyl, —SO₂H, —SO₃H, —OSO₃H, —CONHOH, or —P(OH)OR′, —PO(OH)OR′, —OP(OH)OR′ or —OPO(OH)OR′ where R′ is hydrogen, C₁₋₆ alkyl, C₂₋₆ alkenyl or aryl C₁₋₆ aryl;

R₃ is hydrogen, hydroxy or C₁₋₄ alkoxy; and

R₄ is fluoro, trifluoromethyl, nitro, C₁₋₆ alkyl, C₃₋₇ cycloalkyl, C₂₋₆ alkenyl, C₂₋₆ alkynyl, C₁₋₆ alkylthio, heteroaryl, optionally substituted aryl, optionally substituted aryl C₁₋₆ alkyl, optionally substituted aryl C₂₋₆ alkenyl, optionally substituted aryl C₂₋₆ alkynyl, optionally substituted aryloxy, optionally substituted C₁₋₆ alkoxy, optionally substituted arylthio, optionally substituted aryl C₁₋₄ alylthio or —CONR″R′″, —NR″R′″, —OCONR″R′″ or —SONR″R′″ where R″ and R′″ are each hydrogen, C₁₋₆ alkyl or aryl C₁₋₆ alkyl, or R″ and R′″ together form a C₃₋₇ alkylene ring;

or a salt or ester thereof:

In an embodiment, R¹, R² and R³ are not all hydrogen. In another embodiment, R⁴ is not fluoro when R² and R⁵ are hydrogen and R¹ is hydroxy.

In the above formulae and alkyl group can be straight or branched chain, such as, for example, methyl, ethyl, propyl, isopropyl, butyl and isobutyl, and is preferably methyl or ethyl. A C₂₋₆ alkenyl group includes, for example, vinyl, prop-2-enyl, but-3-enyl, pent-4-enyl and isopropenyl. A preferred alkenyl group is of the formula R—CH═CH— where R is C₁₋₄ alkyl. An alkynyl group includes, for example, prop-2-ynyl, but-3-ynyl, and pent-t-ynyl. A preferred alkynyl group is of the formula:

R—C≡C—

where R is C₁₋₄ alkyl. A C₃₋₇ cycloalkyl group is preferably, for example, cyclopropyl, cyclopentyl or cyclohexyl and these groups may optionally be substituted by one or two methyl substituents.

An aryl group is preferably phenyl or naphthyl, and an optionally substituted phenyl or naphthyl group is optionally substituted with, for example, one or more substituents, preferably 1 to 3 substituents, selected from C₁₋₄ alkyl, especially methyl, C₁₋₄ alkoxy, especially methoxy and ethoxy, carboxy, hydroxy, cyano, halo, especially bromo, chloro and fluoro, trifluoromethyl, nitro, amino, C₁₋₄ acylamino and C₁₋₄ alkylthio. A napthyl group can be 1-naphthyl or 2-naphtyl. When substituted, a phenyl or naphthyl group is preferably substituted by one to three substituents. An aryl C₁₋₆ alkyl group is one such group linked through an alkylene chain, for example, aryl (CH₂)ₙ where n is 1 to 6, and a most preferred example is benzyl. Preferred examples of groups as are follows:

aryloxy—optionally substituted phenoxy:

aryl C₁₋₆ alkoxy—optionally substituted phenylmethyoxy or phenylethoxy;

arylthio—optionally substituted phenylthio;

aryl C₁₋₆ alkylthio—optionally substituted phenylmethythio or phenylethylthio.

A heteroaryl group can be aryl group having one or more hetero atoms in the ring. The term includes fused ring structures. Preferably the heteroaryl group contains one or two hetero atoms selected from oxygen, nitrogen and sulphur. It preferably contains from 5 to 10 carbon atoms, and for example may be of the formula:

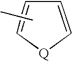

where Q is —O, —S— or —NR—, and R is hydrogen or C₁₋₄ alkyl. Alternatively, a heteroaryl group comprises a benzene fused ring as, for example:

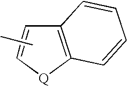

and further heteroaryl groups include:

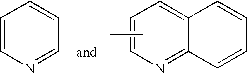

Especially preferred heteroaryl groups are pyrrolyl, thieneyl or furanyl, preferred examples being 2-thieneyl and 2-furanyl, and also pyridyl, in particular 2- and 3-pyridyl.

The group R² is preferably hydrogen, carboxy or tetrazolyl and especially carboxy, and the group R⁴ is preferably C₁₋₆ alkyl, C₂₋₆ alkenyl, optionally substituted phenyl, optionally substituted phenyl C₁₋₆ alkyl, optionally substituted phenoxy, optionally substituted phenylithio or optionally substituted phenyl C₁₋₆ alklthio.

The groups R¹ and R³ are each preferably hydrogen or hydroxy.

Examples of particular antagonists of mGluR5 that are useful in the invention include 2-methyl-6-(phenylethynyl)-pyridine (MPEP), 2-methyl-6-[(1E)-2 phenylethenyl]pyridine. 6-methyl-(phenylazo)-3-pyridinol, (RS)-α-methyl-4-carboxyphenylglycine (MCPG), and analogues and derivatives thereof.

An antagonist of mGluR5 may be used in a method of treatment of the human or animal body. The treatment may be a prophylactic treatment. I particular such antagonists may be used in the tolerance and/or dependence therapy. Antagonists may also be used in the manufacture of a medicament for use in tolerance and/or dependence therapy. The condition of a patient suffering from tolerance and/or dependence can be improved by administration of an antagonist of mGluR5. A therapeutically effective amount of an antagonist of mGluR5 may be given to a human patient in need thereof.

Tolerance and dependence may be treated according to the invention. Tolerance and dependence are separate phenomena.

Tolerance describes an increase in dose needed to produce a given pharmacological effect of a particular substance. In the case of opiates, for example, tolerance develops rapidly.

Dependence involves two separate components, namely physical and psychological dependence. Physical dependence is characterised by a clear-cut abstinence syndrome, such that abrupt withdrawal of a substance (cessation) may lead to, for example, increased irritability or body shakes. The exact nature of the abstinence syndrome is related to the particular substance in question. The invention may be used in the treatment of abstinence syndrome/cessation.

Psychological dependence is more complex than physical dependence and probably more important in the genesis of compulsive substance taking (ie. addiction). Typically, opiate addicts who recover fully from the abstinence syndrome are likely to revert to drug taking later. In animal models of psychological dependence on opiates based on measurement of the potentiality of drugs to act as reinforcers in tests of operant conditioning, the reinforcing effect of the drug outlasts the duration of the physical abstinence syndrome. The invention may used in the treatment of psychological dependence and in the reduction or abolition of the reinforcing effects of drugs.

The invention is applicable to the treatment of many different forms of tolerance or dependence. Tolerance or dependence may be reduced or abolished. Typically, the invention is applicable to the treatment of substance tolerance or substance dependence.

In the context of substance tolerance and dependence, the invention is applicable both to what may be loosely termed “abuse” such as nicotine addiction in the case of smokers or the consumption of other recreational drugs, and to therapeutic usage. For example, therapeutic usage of benzodiazepines and opiates may lead to tolerance to and/or dependence on those drugs. It is clearly advantageous that those consequences of pharmacological therapy effects be ameliorated or abolished.

Thus, an antagonist of a mGluR5 may be used to prevent addiction to a therapeutic pharmaceutical. In such an application, the antagonist of mGluR5 is administered before the said therapeutic pharmaceutical has itself been administered, after the said pharmaceutical has been administered or after withdrawal of the pharmaceutical, or it may be co-administered with the said pharmaceutical.

The invention is also of relevance to the treatment of a wide spectrum of other addiction-related conditions. For example, an antagonist of mGluR5 may be used in the treatment of bulimia nervosa, anorexia nervosa, betting and gambling dependence, sex dependence, sporting activity dependence or obsessive compulsive disorder. An antagonist of mGluR5 may thus be used to treat obsessive and/or compulsive behaviours and the obsessive and/or compulsive components of a variety of disorders such as gambling and/or compulsive sexual activity.

The invention provides treatment of tolerance to and dependence on a number of substances. The invention is particularly useful in the treatment of dependence on nicotine, for example to treat withdrawal effects of smoking cessation. Thus, the invention may be used in the treatment of smoking cessation-related phenomena including craving, depression, anxiety, concentration difficulty and weight gain. Withdrawal symptoms associated with smoking cessation can be reduced.

The invention is also useful in the treatment of cocaine, amphetamine and alcohol addiction. Dependence on cocaine, amphetamines and alcohol, may be reduced or abolished. Tolerance of and dependence on amphetamine-related drugs such as dextroamphetamine, methylamphetamine, methylphenidate and fenfluramine may also be treated using an antagonist of mGluR5.

The invention is further useful in the treatment of opiate tolerance or dependence, in both “abuse” contexts, for example dependence on heroin and pharmaceutical contexts, for example in the prophylaxis of morphine tolerance and/or dependence. Additionally, tolerance and dependence on benzodiazepines, including diazepam and temazepam may be treated by the use of an antagonist of mGluR5.

Antagonists of mGluR5 may be administered in a variety of dosage forms. Thus, they can be administered orally, for example as tablets, troches, lozenges, aqueous or oily suspensions dispersible powders or granules. The antagonists may also be administered parenterally, either subcutaneously, intravenously, intramuscularly, intrasternally, transdermally or by infusion techniques. The inhibitors may also be administered as suppositories. A physician will be able to determine the required route of administration for each particular patient.

The formulation of an antagonist of mGluR5 will depend upon factors such as the nature of the exact antagonist, whether a pharmaceutical or veterinary use is intended, etc. An antagonist of mGluR5 may be formulated for simultaneous, separate or sequential use.

An antagonist of mGluR5 is typically formulated for administration in the present invention with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate, effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

A therapeutically effective amount of an antagonist of mGluR5 is administered to a patient. The dose of an antagonist of mGluR5 may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the specific inhibitor, the age, weight and conditions of the subject to be treated, the type and severity of the degeneration and the frequency and route of administration. Preferably, daily dosage levels are from 5 mg to 2 g.

The invention also provides methods for identifying products which may be used in a method of treatment of the human or animal body by therapy, in particular in the treatment of tolerance or dependence. Such methods essentially comprise determining whether a test product is a mGluR5 antagonist and determining whether an antagonist so-identified can be used in the treatment of substance tolerance or dependence.

Antagonists of mGluR5 are defined above and any suitable assay may be used may be carried out in order to determine whether a test product is an mGluR5 antagonist. Preferably, any assay used will be suitable for high through-put

1. (CANCEL)

2. (CANCEL)

3. (CANCEL)

4. (CANCEL)

5. (CANCEL)

6. (CANCEL)

7. (CANCEL)

8. (amended) A method or treating a mammalian subject suffering from tolerance or dependence, which method comprises administering to the subject a therapeutically effective amount of an antagonist of mGluR5.

9. (amended) A pharmaceutical composition comprising an antagonist of mGluR5and a therapeutic substance, wherein the use of the therapeutic substance in the absence of said antagonist could lead to tolerance of or dependence on the therapeutic substance.

10. (CANCEL)

11. (amended) A method for screening a compound for use in the treatment of tolerance or dependence, comprising:
(a) contacting a test compound with mGluR5 under the conditions that in the absence of the test compound would lead to activity of the said mGluR5; and
determining whether the test compound antagonises mGluR5 activity.

12. (CANCEL)

13. (CANCEL)

14. (CANCEL)

15. (CANCEL)

16. (CANCEL)

17. (CANCEL)

18. (new) A method of treating a subject with abstinence syndrome due to a substance selected from nicotine, an opiate, cocaine, an amphetamine, a benzodiazipine and ethanol,
comprising administering a therapeutically effective amount of an antagonist of mGluR5 to said subject.

19. (new) A method of treating a subject with abstinence syndrome due to a substance selected from an opiate, cocaine, an amphetamine, and a benzodiazipine,
comprising administering a therapeutically effective amount or an antagonist of mGluR5 to said subject.

20. (new) A method of treating a subject with abstinence syndrome due to ethanol,
comprising administering a therapeutically effective amount of an antagonist of mGluR5 to said subject.

21. (new) A method of treating substance dependence in a mammalian subject in need thereof, where said substance is nicotine,
comprising administering a therapeutically effective amount of of an antagonist of mGluR5 to said subject.

22. (new) A method of treating substance dependence in a mammalian subject in need thereof, where said substance is selected from an opiate, cocaine, an amphetamine, and a benzodiazipine,
comprising administering a therapeutically effective amount of of an antagonist of mGluR5 to said subject.

23. (new) A method of treating substance dependence in a mammalian subject in need thereof, where said substance is ethanol,
comprising administering a therapeutically effective amount of of an antagonist of mGluR5 to said subject.

24. (new) A method of treating tolerance to a therapeutic compound, where said compound is selected from opiates, amphetamines, and benzodiazipines,
comprising administering a therapeutically effective amount of of an antagonist of mGluR5 to said subject

25. (new) A method of treating, in a mammalian subject, a condition selected from bulimia nervosa and anorexia, comprising administering a therapeutically effective amount of an antagonist of mGluR5 to said subject.

26. (new) A method of treating, in a mammalian subject, a condition selected from gambling dependence, sex dependence and obsessive compulsive disorder, comprising administering a therapeutically effective amount of an antagonist of mGluR5 to said subject.

27. (new) A pharmaceutical composition comprising pharmaceutically acceptable carrier, an antagonist of mGluR5, and a therapeutic substance selected from opiates, amphetamines, and benzodiazipines.
